(19) United States
(12) Patent Application Publication
(10) Pub. No.: US 2024/0010981 A1
(43) Pub. Date: Jan. 11, 2024

(54) COMPOSITIONS AND METHODS FOR INCREASING STEM CELL FUNCTION

(71) Applicants: SOCIETE DES PRODUITS NESTLE S.A., Vevey (CH); LUDWIG INSTITUTE FOR CANCER RESEARCH LTD, Zurich (CH)

(72) Inventors: NICOLA VANNINI, La Croix (Lutry) (CH); MUKUL GIROTRA, Bridgewater, NJ (US); GEORGE COUKOS, Chexbres (CH)

(21) Appl. No.: 18/246,744

(22) PCT Filed: Sep. 28, 2021
(86) PCT No.: PCT/EP2021/076654

§ 371 Date: Mar. 27, 2023

(30) Foreign Application Priority Data
Sep. 28, 2020 (EP) 20198620.5

(51) Int. Cl. C12N 5/0789; A61K 31/366; A61K 31/706; A61K 31/714

(52) U.S. Cl. CPC C12N 5/0647; A61K 31/366; A61K 31/706; A61K 31/714; C12N 2500/38; C12N 2501/999; C12N 2500/40

(57) ABSTRACT

Use of a composition comprising a combination of a urolithin, a NAD precursor, preferably Nicotinamide Riboside and Vitamin B12 for increasing stem cell function in a population of haematopoietic stem and/or progenitor cells (HSPCs).

### FIELD OF THE INVENTION

The present invention relates to haematopoietic stem and progenitor cells (HSPCs). In particular, the invention relates to composition and methods for increasing stem cell function in haematopoietic stem cells, for example increasing engraftment by a population of HSPCs, and/or increasing capacity for self-renewal and differentiation.

### BACKGROUND TO THE INVENTION

The haematopoietic system is a complex hierarchy of cells of different mature cell lineages. These include cells of the immune system that offer protection from pathogens, cells that carry oxygen through the body and cells involved in wound healing. All these mature cells are derived from a pool of haematopoietic stem cells (HSCs) that are capable of self-renewal and differentiation into any blood cell lineage.

HSCs differ from their committed progeny by relying primarily on anaerobic glycolysis rather than mitochondrial oxidative phosphorylation for energy production (Simsek, T. et al. (2010) Cell Stem Cell 7: 380-90; Takubo, K. et al. (2013) Cell Stem Cell 12: 49-61; Vannini, N. et al. (2016) Nat Commun 7: 13125; Yu, W. M. et al. (2013) Cell Stem Cell 12: 62-74). This distinct metabolic state is believed to protect the HSCs from cellular damage inflicted by reactive oxygen species (ROS) in active mitochondria, thereby maintaining the cells' long-term in vivo function (Chen, C. et al. (2008) J Exp Med 205: 2397-408; Ito, K. et al. (2004) Nature 431: 997-1002; Ito, K. et al. (2006) Nat Med 12: 446-51; Tothova, Z. et al. (2007) Cell 128: 325-39).

Mitochondrial membrane potential, indicated by tetramethylrhodamine methyl ester (TMRM) fluorescence, has previously been used as a surrogate for the metabolic state of cells, and it has been demonstrated that phenotypically defined HSCs have lower mitochondrial membrane potential compared to progenitors (Vannini, N. et al. (2016) Nat Commun 7: 13125). In the same study it was found that artificial lowering of mitochondrial membrane potential, by chemical uncoupling of the mitochondrial electron transport chain, forces the HSCs to maintain their functionality under culture conditions that normally induce rapid differentiation (Vannini, N. et al. (2016) Nat Commun 7: 13125). Importantly, similar mechanisms were observed in human HSCs where artificial lowering of mitochondrial membrane potential by supplementing the culture media with nicotinamide riboside (a NAD and vitamin B3 precursor) resulted in significantly higher levels of engraftment and were capable of sustaining long-term blood production in both primary and secondary recipient humanised mice.

However, there remains a significant need for additional approaches that increase stem cell function in HSCs in vivo and in vitro, in particular approaches that increase engraftment by a population of HSPCs (e.g. during a haematopoietic stem cell transplant procedure) and increase capacity for self-renewal and differentiation by HSCs.

### SUMMARY OF THE INVENTION

The applicant has found that a combination of urolithin A (UroA) with a Nicotinamide Adenine Dinucleotide (NAD+) precursor, preferably Nicotinamide riboside and Vitamin B12 (preferably methylcobalamine) had a synergistic effect on ameliorating haematopoietic stem cell (HSC) function, such as through increasing engraftment and self-renewal.

While not wishing to be bound by theory, the increased stem cell function may be achieved via modulation of mitochondrial membrane potential through mitophagy induction in cells exposed to said combination.

In one aspect, the invention provides use of a composition comprising a combination of a urolithin, a NAD+ precursor and Vitamin B12 for increasing stem cell function in a population of haematopoietic stem and/or progenitor cells (HSPCs).

In some embodiments, the use is in vitro use. In some embodiments, the use is ex vivo use.

In some embodiments, the population is an isolated population of HSPCs.

In some embodiments, the HSPCs have a CD34+ phenotype.

In some embodiments, the HSPCs have a CD34+CD38−phenotype.

In another aspect, the invention provides a composition comprising a combination of a urolithin, a NAD+ precursor and Vitamin B12 for use in increasing haematopoietic stem cell function.

In some embodiments, the composition comprising a combination of a urolithin, a NAD+ precursor and Vitamin B12 is for use in increasing haematopoietic stem cell function in a subject.

In some embodiments, the stem cell function comprises engraftment. In some embodiments, the stem cell function comprises self-renewal. In some embodiments, the stem cell function comprises differentiation.

In some embodiments, the stem cell function is engraftment. In some embodiments, the stem cell function is self-renewal. In some embodiments, the stem cell function is differentiation.

In another aspect, the invention provides a composition comprising a combination of a urolithin, a NAD+ precursor and Vitamin B12 for use in increasing blood cell levels in a subject.

In another aspect, the invention provides a composition comprising a combination of a urolithin, a NAD+ precursor and Vitamin B12 for use in the treatment or prevention of (a) anaemia, leukopenia and/or thrombocytopenia; (b) an infection; and/or (c) cancer in a subject.

In another aspect, the invention provides a composition comprising a combination of a urolithin, a NAD+ precursor and Vitamin B12 for use in the treatment or prevention of anaemia, leukopenia and/or thrombocytopenia.

In another aspect, the invention provides a composition comprising a combination of a urolithin, a NAD+ precursor and Vitamin B12 for use in the treatment or prevention of an infection.

In another aspect, the invention provides a composition comprising a combination of a urolithin, a NAD+ precursor and Vitamin B12 for use in the treatment or prevention of cancer.

In some embodiments, the cancer is a haematological cancer. In some embodiments, the cancer is leukaemia, lymphoma or myeloma.

In preferred embodiments, the urolithin is urolithin A.

In a preferred embodiment, the NAD+ precursor is selected from the group consisting Nicotinic Acid, Nicotinamide, Nicotinamide Riboside (NR), Reduced Nicotinamide riboside (NRH), Nicotinamide Mononucleotide (NMN), Nicotinic acid mononucleotide, Nicotinic acid riboside, and mixtures thereof.

In a preferred embodiment, the combination comprises Urolithin A, Nicotinamide riboside and Vitamin B12.

In some embodiments, the composition comprising a combination of a urolithin, a NAD+ precursor and Vitamin B12 is administered to a subject enterally or parenterally, preferably enterally. In preferred embodiments, the combination is administered to a subject orally.

In some embodiments, the composition comprising a combination of a urolithin, a NAD+ precursor and Vitamin B12 is in the form of a pharmaceutical or nutritional composition.

In some embodiments, the composition comprising a combination of a urolithin, a NAD+ precursor and Vitamin B12 is in the form of a food product, food supplement, nutraceutical, food for special medical purpose (FSMP), nutritional supplement, dairy-based drink, low-volume liquid supplement or meal replacement beverage.

In some embodiments, a subject has or is at risk of having subnormal amounts of haematopoietic cells, for example erythrocytes, leukocytes and/or platelets.

In some embodiments, a subject has or is at risk of having anaemia, leukopenia and/or thrombocytopenia.

In some embodiments, a subject has undergone an intervention selected from the group consisting of a haematopoietic stem cell transplant; a bone marrow transplant; myeloablative conditioning; chemotherapy; radiotherapy; and surgery.

In some embodiments, a subject is an immune-compromised subject.

In some embodiments, the subject is 3-4 weeks post-intervention.

In some embodiments, a subject is a human or non-human mammal, preferably a human, optionally a human adult, child or infant.

In some embodiments, the Urolithin, a NAD+ precursor and Vitamin B12 are administered to the subject simultaneously, sequentially or separately, preferably simultaneously.

In some embodiments, the composition comprising a combination of a urolithin, a NAD+ precursor and Vitamin B12 is in a combined preparation for simultaneous, separate or sequential use with an agent selected from the group consisting of a G-CSF analogue, a TPO receptor analogue, SCF, TPO, Flt3-L, FGF-1, IGF1, IGFBP2, IL-3, IL-6, G-CSF, M-CSF, GM-CSF, EPO and combinations thereof.

In another aspect, the invention provides a method of expanding an isolated population of haematopoietic stem and/or progenitor cells (HSPCs) comprising contacting the population with a composition comprising a combination of a urolithin, a NAD+ precursor and Vitamin B12.

In some embodiments, the contacting comprises culturing the population in the presence of the composition comprising a combination of a urolithin, a NAD+ precursor and Vitamin B12.

In some embodiments, the method comprises the steps:
(a) providing a population of HSPCs;
(b) optionally culturing the population of HSPCs, preferably in a HSPC expansion or maintenance culture medium;
(c) optionally isolating a sub-population of HSPCs characterised by low mitochondrial membrane potential; and
(d) contacting the population of (a) or (b), or the sub-population of (c) with a composition comprising a combination of a urolithin, a NAD+ precursor and Vitamin B12.

In some embodiments, the population provided in step (a) is obtained from bone marrow, mobilised peripheral blood or umbilical cord blood.

In some embodiments, the product of step (d) is enriched in cells having long-term multi-lineage blood reconstitution capability.

In another aspect, the invention provides a population of haematopoietic stem and/or progenitor cells (HSPCs) obtainable by the method of the invention.

In another aspect, the invention provides a pharmaceutical composition comprising the population of haematopoietic stem and/or progenitor cells (HSPCs) of the invention.

In another aspect, the invention provides a cell culture medium comprising a composition comprising a combination of a urolithin, Nicotinamide Riboside and Vitamin B12.

In preferred embodiments, the urolithin is urolithin A.

In a preferred embodiment, the NAD+ precursor is selected from the group consisting Nicotinic Acid, Nicotinamide, Nicotinamide Riboside (NR), Reduced Nicotinamide riboside (NRH), Nicotinamide Mononucleotide (NMN), Nicotinic acid mononucleotide, Nicotinic acid riboside, and mixtures thereof.

In a preferred embodiment, the combination comprises Urolithin A, Nicotinamide riboside and Vitamin B12.

In some embodiments, the culture medium is a haematopoietic stem and/or progenitor cell (HSPC) culture medium.

In some embodiments, the culture medium is an expansion or maintenance culture medium.

In another aspect, the invention provides a method of engrafting a subject with haematopoietic stem and/or progenitor cells (HSPCs) comprising contacting an isolated population of HSPCs with a composition comprising a combination of a urolithin, NAD+ precursor and Vitamin B12, and administering the population of HSPCs to the subject in need thereof.

In another aspect, the invention provides a method of increasing haematopoietic stem cell function comprising contacting a population of haematopoietic stem and/or progenitor cells (HSPCs) with a composition comprising a combination of a urolithin, NAD+ precursor and Vitamin B12.

In another aspect, the invention provides a method of increasing haematopoietic stem cell function in a subject comprising contacting a population of haematopoietic stem and/or progenitor cells (HSPCs) with a composition comprising a combination of a urolithin, NAD+ precursor and Vitamin B12, and administering the population of HSPCs to the subject in need thereof.

In another aspect, the invention provides a method of increasing haematopoietic stem cell engraftment comprising contacting a population of haematopoietic stem and/or progenitor cells (HSPCs) with a composition comprising a combination of a urolithin, NAD+ precursor and Vitamin B12. In another aspect the invention provides a method of increasing haematopoietic stem cell self-renewal comprising contacting a population of haematopoietic stem and/or progenitor cells (HSPCs) with a composition comprising a combination of a urolithin, NAD+ precursor and Vitamin B12. In another aspect the invention provides a method of increasing haematopoietic stem cell differentiation comprising contacting a population of haematopoietic stem and/or progenitor cells (HSPCs) with a composition comprising a combination of a urolithin, NAD+ precursor and Vitamin B12. In some embodiments, the engraftment, self-renewal and/or differentiation are increased in a subject and the method further comprises administering the population of HSPCs to the subject in need thereof.

In another aspect, the invention provides a method of (a) increasing blood cell levels; (b) treating or preventing anaemia, leukopenia and/or thrombocytopenia; (c) treating or preventing an infection: and/or (d) treating or preventing cancer in a subject comprising contacting a population of haematopoietic stem and/or progenitor cells (HSPCs) with a composition comprising a combination of a urolithin, a NAD+ precursor and Vitamin B12, and administering the population of HSPCs to the subject in need thereof.

In another aspect, the invention provides a method of increasing blood cell levels comprising contacting a population of haematopoietic stem and/or progenitor cells (HSPCs) with a composition comprising a combination of a urolithin, a NAD+ precursor and Vitamin B12.

In another aspect, the invention provides a method of treating or preventing anaemia, leukopenia and/or thrombocytopenia comprising contacting a population of haematopoietic stem and/or progenitor cells (HSPCs) with a composition comprising a combination of a urolithin, a NAD+ precursor and Vitamin B12.

In another aspect, the invention provides a method of treating or preventing an infection comprising contacting a population of haematopoietic stem and/or progenitor cells (HSPCs) with a composition comprising a combination of a urolithin, a NAD+ precursor and Vitamin B12.

In another aspect, the invention provides a method of treating or preventing cancer comprising contacting a population of haematopoietic stem and/or progenitor cells (HSPCs) with a composition comprising a combination of a urolithin, a NAD+ precursor and Vitamin B12.

In some embodiments, the method is an ex vivo method. In some embodiments, the method is an in vivo method.

In some embodiments, the population is an isolated population of HSPCs. In some embodiments, the method further comprises administering the population of HSPCs to a subject in need thereof.

In another aspect, the invention provides a method of increasing haematopoietic stem cell function comprising administering a composition comprising a combination of a urolithin, a NAD+ precursor and Vitamin B12 to a subject in need thereof.

In another aspect, the invention provides a method of increasing haematopoietic stem cell engraftment comprising administering a composition comprising a combination of a urolithin, a NAD+ precursor and Vitamin B12 to a subject in need thereof. In another aspect the invention provides a method of increasing haematopoietic stem cell self-renewal comprising administering a composition comprising a combination of a urolithin, a NAD+ precursor and Vitamin B12 to a subject in need thereof. In another aspect the invention provides a method of increasing haematopoietic stem cell differentiation comprising administering a composition comprising a combination of a urolithin, a NAD+ precursor and Vitamin B12 to a subject in need thereof.

In another aspect, the invention provides a method of increasing blood cell levels comprising administering a composition comprising a combination of a urolithin, a NAD+ precursor and Vitamin B12 to a subject in need thereof.

In another aspect, the invention provides a method of treating or preventing anaemia, leukopenia and/or thrombocytopenia comprising administering a composition comprising a combination of a urolithin, a NAD+ precursor and Vitamin B12 to a subject in need thereof.

In another aspect, the invention provides a method of treating or preventing an infection comprising administering a composition comprising a combination of a urolithin, a NAD+ precursor and Vitamin B12 to a subject in need thereof.

In another aspect, the invention provides a method of treating or preventing cancer comprising administering a composition comprising a combination of a urolithin, NAD+ precursor and Vitamin B12 to a subject in need thereof.

### DESCRIPTION OF THE DRAWINGS

FIG. 1

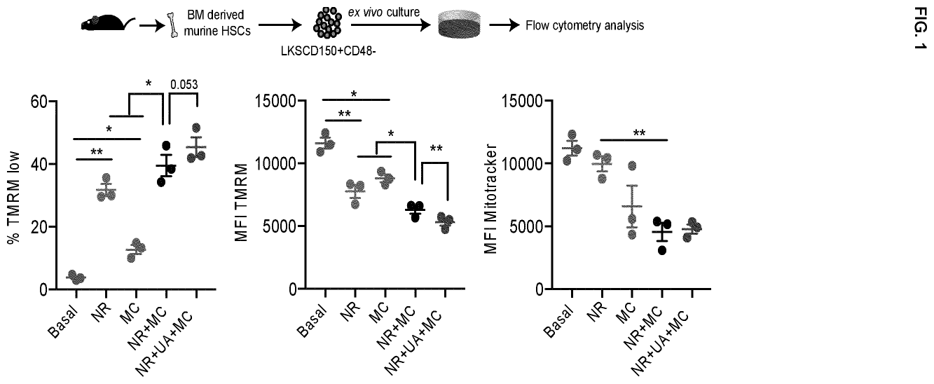

Combination of UroA, NR and Vitamin B12 induces lowering of mitochondrial membrane potential. A) Bone marrow derived murine HSCs cultured in basal media (control) supplemented with NR, Vit B12 (MC), NR+MC and NR+UroA+MC (concentrations; NR: 500 uM, UroA: 20 uM, Vit B12 (MC): 100 uM. NR was replenished every 24 hours. The proportion of cells in the TMRM low gate increases and the MFI TMRM decreases in the NR and MC conditions alone (left and middle panel). Additionally, combination of NR+MC was significantly different from NR or MC alone. The triple combination of UroA+MC+NR showed the strongest effect and was significantly different from the duo combinations. Mitochondrial mass (measured by Mitotracker) (right panel) decreases significantly with combination of NR+MC and UroA+MC+NR compared to Basal or only NR in culture.

### DETAILED DESCRIPTION OF THE INVENTION

The terms “comprising”, “comprises” and “comprised of” as used herein are synonymous with “including” or “includes”; or “containing” or “contains” and are inclusive or open-ended and do not exclude additional, non-recited members, elements or steps. The terms “comprising”, “comprises” and “comprised of” also include the term “consisting of”.

Haematopoietic Stem Cells

A stem cell is able to differentiate into many cell types. A cell that is able to differentiate into all cell types is known as totipotent. In mammals, only the zygote and early embryonic cells are totipotent. Stem cells are found in most, if not all, multicellular organisms. They are characterised by the ability to renew themselves through mitotic cell division and differentiate into a diverse range of specialised cell types. The two broad types of mammalian stem cells are embryonic stem cells that are isolated from the inner cell mass of blastocysts, and adult stem cells that are found in adult tissues. In a developing embryo, stem cells can differentiate into all of the specialised embryonic tissues. In adult organisms, stem cells and progenitor cells act as a repair system for the body, replenishing specialised cells, but also maintaining the normal turnover of regenerative organs, such as blood, skin or intestinal tissues.

Haematopoietic stem cells (HSCs) are multipotent stem cells that may be found, for example, in peripheral blood, bone marrow and umbilical cord blood. HSCs are capable of self-renewal and differentiation into any blood cell lineage. They are capable of recolonising the entire immune system, and the erythroid and myeloid lineages in all the haematopoietic tissues (such as bone marrow, spleen and thymus). They provide for life-long production of all lineages of haematopoietic cells.

Haematopoietic progenitor cells have the capacity to differentiate into a specific type of cell. In contrast to stem cells however, they are already far more specific: they are pushed to differentiate into their “target” cell. A difference between stem cells and progenitor cells is that stem cells can replicate indefinitely, whereas progenitor cells can only divide a limited number of times. Haematopoietic progenitor cells can be rigorously distinguished from HSCs only by functional in vivo assay (i.e. transplantation and demonstration of whether they can give rise to all blood lineages over prolonged time periods).

A differentiated cell is a cell which has become more specialised in comparison to a stem cell or progenitor cell. Differentiation occurs during the development of a multicellular organism as the organism changes from a single zygote to a complex system of tissues and cell types. Differentiation is also a common process in adults: adult stem cells divide and create fully-differentiated daughter cells during tissue repair and normal cell turnover. Differentiation dramatically changes a cell's size, shape, membrane potential, metabolic activity and responsiveness to signals. These changes are largely due to highly-controlled modifications in gene expression. In other words a differentiated cell is a cell which has specific structures and performs certain functions due to a developmental process which involves the activation and deactivation of specific genes. Here, a differentiated cell includes differentiated cells of the haematopoietic lineage such as monocytes, macrophages, neutrophils, basophils, eosinophils, erythrocytes, megakaryocytes/platelets, dendritic cells, T-cells, B-cells and NK-cells. For example, differentiated cells of the haematopoietic lineage can be distinguished from stem cells and progenitor cells by detection of cell surface molecules which are not expressed or are expressed to a lesser degree on undifferentiated cells. Examples of suitable human lineage markers include CD33, CD13, CD14, CD15 (myeloid), CD19, CD20, CD22, CD79a (B), CD36, CD71, CD235a (erythroid), CD2, CD3, CD4, CD8 (T), CD56 (NK).

HSC Source

In some embodiments, haematopoietic stem cells are obtained from a tissue sample.

For example, HSCs can be obtained from adult and foetal peripheral blood, umbilical cord blood, bone marrow, liver or spleen. They may be obtained after mobilisation of the cells in vivo by means of growth factor treatment.

Mobilisation may be carried out using, for example, G-CSF, plerixaphor or combinations thereof. Other agents, such as NSAIDs, CXCR2 ligands (Grobeta) and dipeptidyl peptidase inhibitors may also be useful as mobilising agents.

With the availability of the stem cell growth factors GM-CSF and G-CSF, most haematopoietic stem cell transplantation procedures are now performed using stem cells collected from the peripheral blood, rather than from the bone marrow. Collecting peripheral blood stem cells provides a bigger graft, does not require that the donor be subjected to general anaesthesia to collect the graft, results in a shorter time to engraftment and may provide for a lower long-term relapse rate.

Bone marrow may be collected by standard aspiration methods (either steady-state or after mobilisation), or by using next-generation harvesting tools (e.g. Marrow Miner).

In addition, HSCs may be derived from induced pluripotent stem cells.

HSC Characteristics

HSCs are typically of low forward scatter and side scatter profile by flow cytometric procedures. Some are metabolically quiescent, as demonstrated by Rhodamine labelling which allows determination of mitochondrial activity. Human HSCs may comprise certain cell surface markers such as CD34, CD45, CD133, CD90 and CD49f. They may also be defined as cells lacking the expression of the CD38 and CD45RA cell surface markers. However, expression of some of these markers is dependent upon the developmental stage and tissue-specific context of the HSC. Some HSCs called “side population cells” exclude the Hoechst 33342 dye as detected by flow cytometry. Thus, HSCs have descriptive characteristics that allow for their identification and isolation.

Negative Markers

CD38 is the most established and useful single negative marker for human HSCs.

Human HSCs may also be negative for lineage markers such as CD2, CD3, CD14, CD16, CD19, CD20, CD24, CD36, CD56, CD66b, CD271 and CD45RA. However, these markers may need to be used in combination for HSC enrichment.

By “negative marker”, it is to be understood that human HSCs lack the expression of these markers.

Positive Markers

CD34 and CD133 are the most useful positive markers for HSCs.

Some HSCs are also positive for lineage markers such as CD90, CD49f and CD93. However, these markers may need to be used in combination for HSC enrichment.

By “positive marker”, it is to be understood that human HSCs express these markers.

In some embodiments, the HSCs have a CD34+ phenotype.

In some embodiments, the HSCs have a CD34+CD38− phenotype.

Further separations may be carried out to obtain, for example, CD34+CD38−CD45RA−CD90+CD49f+ cells.

Stem Cell Function

The term “stem cell function” as used herein refers to characteristics of a cell that are typically associated with a stem cell, for example the ability to differentiate into specific cellular lineages and/or the ability to self renew.

In one embodiment, the stem cell function comprises engraftment, self-renewal and/or differentiation.

In some embodiments, the stem cell function comprises engraftment. In some embodiments, the stem cell function comprises self-renewal. In some embodiments, the stem cell function comprises differentiation.

In one embodiment, the stem cell function is engraftment, self-renewal and/or differentiation.

In some embodiments, the stem cell function is engraftment. In some embodiments, the stem cell function is self-renewal. In some embodiments, the stem cell function is differentiation.

The term “engraftment” as used herein refers to the ability of the haematopoietic stem and/or progenitor cells to populate and survive in a subject following their transplantation, i.e. in the short and/or long term after transplantation. For example, engraftment may refer to the number and/or percentages of haematopoietic cells descended from the transplanted haematopoietic stem and/or progenitor cells (e.g. graft-derived cells) that are detected about 1 day to 24 weeks, 1 day to 10 weeks, or 1-30 days or 10-30 days after transplantation. In some embodiments, engraftment is assessed at about 1, 2, 3, 4, 5, 6, 7, 8, 9, 10, 15, 20, 25 or 30 days after transplantation. In other embodiments, engraftment is assessed at about 4, 5, 6, 7, 8, 9, 10, 11, 12, 13, 14, 15, 16, 17, 18, 19, 20, 21, 22, 23 or 24 weeks after transplantation. In other embodiments, engraftment is assessed at about 16-24 weeks, preferably 20 weeks, after transplantation.

Engraftment may be readily analysed by the skilled person. For example, the transplanted haematopoietic stem and/or progenitor cells may be engineered to comprise a marker (e.g. a reporter protein, such as a fluorescent protein), which can be used to quantify the graft-derived cells. Samples for analysis may be extracted from relevant tissues and analysed ex vivo (e.g. using flow cytometry).

The term “self renewal” as used herein refers to the ability of a cell to undergo multiple cycles of cell division while maintaining an undifferentiated state.

Cell numbers and/or percentages in certain states (e.g. live, dead or apoptotic cells) may be quantified using any of a number of methods known in the art, including use of haemocytometers, automated cell counters, flow cytometers and fluorescence activated cell sorting machines. These techniques may enable distinguishing between live, dead and/or apoptotic cells. In addition or in the alternative, apoptotic cells may be detected using readily available apoptosis assays (e.g. assays based on the detection of phosphatidylserine (PS) on the cell membrane surface, such as through use of Annexin V, which binds to exposed PS; apoptotic cells may be quantified through use of fluorescently-labelled Annexin V), which may be used to complement other techniques.

Isolation and Enrichment of Populations of Cells

Populations of cells, such as haematopoietic stem and/or progenitor cells (HSPCs), are disclosed herein. In some embodiments, the population of cells is an isolated population of cells.

The term “isolated population” as used herein refers to a population of cells that is not comprised within the body. An isolated population of cells may have been previously removed from a subject. An isolated population of cells may be cultured and manipulated ex vivo or in vitro using standard techniques known in the art. An isolated population of cells may later be reintroduced into a subject. Said subject may be the same subject from which the cells were originally isolated or a different subject.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (such as CD34) may be purified from a starting population of cells. Alternatively, or in addition, a population of cells that does not express another marker (such as CD38) may be purified.

The term “enriching” as used herein refers to an increase in the concentration of a type of cells within a population. The concentration of other types of cells may be concomitantly reduced.

Purification or enrichment may result in the population of cells being substantially pure of other types of cell.

Purifying or enriching for a population of cells expressing a specific marker (e.g. CD34 or CD38) may be achieved by using an agent that binds to that marker, preferably substantially specifically to that marker.

An agent that binds to a cellular marker may be an antibody, for example an anti-CD34 or anti-CD38 antibody.

The term “antibody” as used herein refers to complete antibodies or antibody fragments capable of binding to a selected target, and including Fv, ScFv, F(ab′) and F(ab′)₂, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification, or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such as biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry.

Clinical grade separation may be performed, for example, using the CliniMACS® system (Miltenyi). This is an example of a closed-circuit magnetic bead-based separation technology.

It is also envisaged that dye exclusion properties (e.g. side population or rhodamine labelling) or enzymatic activity (e.g. ALDH activity) may be used to enrich for HSCs.

Composition

When contacted with an in vitro culture of T cells, the composition of the invention may be used in any form suitable for in vitro cell culture (e.g. a non-toxic form). When administered to a subject, the composition of the invention may be used in any form suitable for ingestion by animals, preferably humans (e.g. are non-toxic).

The composition may be used, for example in compositions such as nutritional compositions, in any appropriate amount. The skilled person will be able to determine appropriate amounts depending on the desired dosage of the agent. Dosages may depend on factors such as the age, size and health status of the subject to whom they are administered, on lifestyle, as well as on genetic heritage. Dosages may be in line with the recommended daily intakes (RDA) developed by organisations such as the Food and Nutrition Board of the National Academy of Sciences.

NAD+ Precursor

The NAD+ precursor according to the present invention is selected from the group consisting of Nicotinic Acid (Niacin), Nicotinamide (Niacinamide), Nicotinamide Riboside (NR), Reduced Nicotinamide Riboside (NRH), Beta-Nicotinamide Mononucleotide (NMN), Nicotinic acid mononucleotide, Nicotinic acid riboside, a food extract enriched in at least one of these compounds, e.g., a food extract enriched in Nicotinamide adenine dinucleotide (NAD), and mixtures thereof. As used herein, “nicotinamide riboside” includes L-valine and L-phenylalanine esters of nicotinamide riboside.

In a preferred embodiment, the NAD+ precursor is Nicotinamide riboside.

Nicotinamide Riboside

Nicotinamide riboside (NR) is a pyridine-nucleoside form of vitamin B3, which is a precursor to nicotinamide adenine dinucleotide (NAD).

Nicotinamide riboside has the structure:

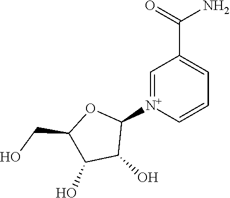 [SMILES: NC(=O)c1ccc[n+]([C@@H]2O[C@H](CO)[C@@H](O)[C@H]2O)c1]

In some embodiments, the T cells are contacted with the NR at a NR concentration of 1-10, 1-5, 1-2.5 or 1-2 mM. In other embodiments, the T cells are contacted with the NR at a NR concentration of 1, 2, 3, 4, 5, 6, 7, 8, 9 or 10 mM, preferably 2 mM.

The nicotinamide riboside or NAD+ precursor can be administered in an amount of about mg/day to about 2000 mg/day, preferably about 0.001 mg/day to about 1000 mg/day, more preferably about 0.001 mg/day to about 750 mg/day, even more preferably about 0.001 mg/day to about 500 mg/day, most preferably about 0.001 mg/day to about 250 mg/day, for example about 0.001 mg/day to about 100 mg/day, about 0.001 mg/day to about 75 mg/day, about 0.001 mg/day to about 50 mg/day, about 0.001 mg/day to about 25 mg/day, about 0.001 mg/day to about 10 mg/day, or about 0.001 mg/day to about 1 mg/day. Of course, the daily dose can be administered in portions at various hours of the day. However, in any given case, the amount of compound administered will depend on such factors as the solubility of the active component, the formulation used, subject condition (such as weight), and/or the route of administration. For example, the daily doses of nicotinamide riboside disclosed above are non-limiting and, in some embodiments, may be different; in particular, the compositions disclosed herein can be utilized as an acute care food for special medical purposes (FSMP) and contain up to about 2.0 mg nicotinamide riboside/day.

Vitamin B12

Vitamin B12 (also known as cobalamine) is a class of cobalt-containing hydrosoluble vitamins which cannot be synthesised by the human body and must therefore be acquired from food or synthesised by the gut microbiota.

The vitamin B12 pool in the human body is composed of several forms: cyanocobalamin, which is inactive and requires conversion for activity, and methylcobalamin and adenosylcobalamin, which are the metabolically active forms of vitamin B12.

Two enzymes are known to rely on vitamin B12 as a cofactor: methionine synthase and methylmalonylCoA mutase. Methionine synthase is a cytoplasmic enzyme relying on methylcobalamine to convert homocysteine to methionine. It thereby plays a critical role in providing S-adenosylmethionine (SAM) as a methylation donor and preventing the toxic accumulation of homocysteine. Low SAM levels and high homocysteine levels observed upon severe vitamin B12 deficiency impair myelination of peripheral nerves and the spinal cord. Methionine synthase also catalyses the activation of 5-methyl-tetrahydrofolate into the bioactive tetrahydrofolate, which is required for 1-carbon metabolism and DNA synthesis, and thus for efficient red blood cell proliferation. MethylmalonylCoA mutase is a mitochondrial enzyme relying on adenosyl-cobalamine to convert methyl-malonylCoA to succinylCoA, which subsequently enters the TCA cycle. It is implicated in the degradation of branched-chain amino acids and odd-chain length fatty acids, and is essential during embryonic life to control neurological development, but is not vital in adult life

The vitamin B12 of the invention may be in the form of, for example, vitamin B12 itself, the semi-synthetic derivative cyanocobalamin, hydroxocobalamin, methylcobalamin and/or adenosylcobalamin. Methylcobalamin may be particularly effective.

In some embodiments, the T cells are contacted with the vitamin B12 at a vitamin B12 concentration of 10-100 μM, 10-75 μM or 10-50 μM. In other embodiments, the T cells are contacted with the vitamin B12 at a vitamin B12 concentration of 25-100 μM, 25-75 μM or 25-50 μM. In other embodiments, the T cells are contacted with the vitamin B12 at a vitamin B12 concentration of 1, 2, 3, 4, 5, 6, 7, 8, 9, 10, 15, 20, 25, 30, 35, 40, 45, 50, 55, 60, 65, 70, 75, 80, 85, 90, 95 or 100 μM, preferably 50 μM.

In some embodiments, the vitamin B12 is administered to a subject at 0.1 to 40 times the recommended daily requirement (RDA) of Vitamin B12 per day, e.g. 1 to 10 times the recommended daily requirement (RDA) of Vitamin B12 per day.

The Vitamin B12 may thus be administered in a daily dose of about 10, 20, 30 or 40 times the RDA of the Vitamin B12 per day. Preferably, the daily dose provides 10 to 40, more preferably 10 to 30 or even more preferably 10 to 25 times the RDA of the Vitamin B12 per day, most preferably about 12 to 21 times the RDA of the Vitamin B12 per day.

The United States RDA of Vitamin B12 is 2.4 micrograms daily for humans of age 14 years and older, so such individuals may be administered a daily dose that provides about 0.002 mg to about 0.4 mg of Vitamin B12 per day, preferably 0.02 mg to 0.07 mg of Vitamin B12 per day, more preferably 0.03 mg to 0.05 mg of Vitamin B12 per day.

Urolithins

Urolithins are metabolites of dietary ellagic acid derivatives, such as ellagitannins, and are produced in the human gut by gut bacteria.

Ellagitannins are a class of antioxidant polyphenols found in several fruits, particularly pomegranate, strawberries, raspberries and walnuts. Although the absorption of ellagitannins is extremely low, they are rapidly metabolised by the gut microbiota of the large intestine into urolithins.

Due to their superior absorption, urolithins are believed to be the bioactive molecules mediating the effects of ellagitannins. To that end, for example, urolithins were previously shown to have antioxidant and anti-inflammatory properties.

Example urolithins include urolithin A (3,8-dihydroxyurolithin), urolithin B (3-hydroxyurolithin), and urolithin D (3,4,8,9-tetrahydroxyurolithin), urolithin A glucuronide and urolithin B glucuronide.

Urolithin A (UroA) has the structure:

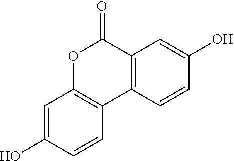 [SMILES: O=c1oc2cc(O)ccc2c2ccc(O)cc12]

In some embodiments, the HSPCs are contacted with the urolithin at a urolithin concentration of 5-250 μM, 5-200 μM, 5-150 μM, 5-100 μM or 5-50 μM. In other embodiments, the HSPCs are contacted with the urolithin at a urolithin concentration of 10-250 μM, 10-200 μM, 10-150 μM, 10-100 μM or 10-50 μM. In other embodiments, the HSPCs are contacted with the urolithin at a urolithin concentration of 20-250 μM, 20-200 μM, 20-150 μM, 20-100 μM or 20-50 μM. In other embodiments, the HSPCs are contacted with the urolithin at a urolithin concentration of 5, 10, 15, 20, 25, 30, 35, 40, 45, 50, 55, 60, 65, 70, 75, 100, 125, 150, 175, 200, 225 or 250 μM.

In preferred embodiments, the HSPCs are contacted with the urolithin at a urolithin concentration of 20-50 μM.

The Urolithin may thus be administered in a total amount of about 0.2-150 milligram (mg) of urolithin per kilogram (kg) of body weight of the subject. Preferably, the at least one urolithin is administered in a daily dose equal or equivalent to 2-120 mg of urolithin per kg body weight of the subject, more preferably 4-90 mg of urolithin per kg body weight of the subject, most preferably 8-30 mg of urolithin per kg body weight of the subject. Any given dose may be given as a single dose or as divided doses.

The urolithin of the invention can be present as a salt or ester, in particular a pharmaceutically-acceptable salt or ester.

Pharmaceutically-acceptable salts of the agents of the invention include suitable acid addition or base salts thereof. A review of suitable pharmaceutical salts may be found in Berge et al. (1977) J Pharm Sci 66: 1-19.

The invention also includes where appropriate all enantiomers and tautomers of the agents. The skilled person will recognise compounds that possess optical properties (e.g. one or more chiral carbon atoms) or tautomeric characteristics. The corresponding enantiomers and/or tautomers may be isolated/prepared by methods known in the art.

Pharmaceutical and Nutritional Compositions

In some embodiments, the composition comprising a combination of a urolithin, Nicotinamide Riboside and Vitamin B12 is in the form of a pharmaceutical composition.

The pharmaceutical composition may further comprise a pharmaceutically acceptable carrier, diluent or excipient.

In some embodiments, the haematopoietic stem and/or progenitor cells (HSPCs) are in the form of a pharmaceutical composition.

The cells of the invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of the cell therapy product is preferably performed in compliance with FACT-JACIE International Standards for cellular therapy.

In some embodiments, the composition comprising a combination of a urolithin, Nicotinamide Riboside and Vitamin B12 is in the form of a nutritional composition.

In some embodiments, the composition comprising a combination of a urolithin, Nicotinamide Riboside and Vitamin B12 is in the form of a food product, food supplement, nutraceutical, food for special medical purpose (FSMP), nutritional supplement, dairy-based drink, low-volume liquid supplement or meal replacement beverage. In some embodiments, the composition is an infant formula.

In some embodiments, the composition comprising a combination of a urolithin, Nicotinamide Riboside and Vitamin B12 is in the form of a food additive or a medicament.

A food additive or a medicament may be in the form of tablets, capsules, pastilles or a liquid for example. Food additives or medicaments are preferably provided as sustained release formulations, allowing a constant supply of the urolithin or precursor thereof for prolonged times.

The composition may be selected from the group consisting of milk-powder based products; instant drinks; ready-to-drink formulations; nutritional powders; nutritional liquids; milk-based products, in particular yoghurts or ice cream; cereal products; beverages; water; coffee; cappuccino; malt drinks; chocolate flavoured drinks; culinary products; soups; tablets; and/or syrups.

The composition may further contain protective hydrocolloids (such as gums, proteins, modified starches), binders, film forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials.

Further, the composition may contain an organic or inorganic carrier material suitable for oral or enteral administration as well as vitamins, minerals trace elements and other micronutrients in accordance with the recommendations of government bodies such as the USRDA.

The composition of the invention may contain a protein source, a carbohydrate source and/or a lipid source.

Any suitable dietary protein may be used, for example animal proteins (such as milk proteins, meat proteins and egg proteins); vegetable proteins (such as soy protein, wheat protein, rice protein and pea protein); mixtures of free amino acids; or combinations thereof. Milk proteins such as casein and whey, and soy proteins are particularly preferred.

If the composition includes a fat source, the fat source preferably provides 5% to 40% of the energy of the formula; for example 20% to 30% of the energy. DHA may be added. A suitable fat profile may be obtained using a blend of canola oil, corn oil and high-oleic acid sunflower oil.

A source of carbohydrates may more preferably provide between 40% to 80% of the energy of the composition. Any suitable carbohydrate may be used, for example sucrose, lactose, glucose, fructose, corn syrup solids, maltodextrins and mixtures thereof.

Hematopoietic Stem Cell Transplantation

The invention provides a population of haematopoietic stem and/or progenitor cells prepared according to a method of the invention for use in therapy.

The use may be as part of a haematopoietic stem cell transplantation procedure.

Hematopoietic stem cell transplantation (HSCT) is the transplantation of blood stem cells derived from the bone marrow (in this case known as bone marrow transplantation) or blood. Stem cell transplantation is a medical procedure in the fields of haematology and oncology, most often performed for people with diseases of the blood or bone marrow, or certain types of cancer.

Many recipients of HSCTs are multiple myeloma or leukaemia patients who would not benefit from prolonged treatment with, or are already resistant to, chemotherapy. Candidates for HSCTs include paediatric cases where the patient has an inborn defect such as severe combined immunodeficiency or congenital neutropenia with defective stem cells, and also children or adults with aplastic anaemia who have lost their stem cells after birth. Other conditions treated with stem cell transplants include sickle-cell disease, myelodysplastic syndrome, neuroblastoma, lymphoma, Ewing's Sarcoma, Desmoplastic small round cell tumour and Hodgkin's disease. More recently non-myeloablative, or so-called “mini transplant”, procedures have been developed that require smaller doses of preparative chemotherapy and radiation. This has allowed HSCT to be conducted in the elderly and other patients who would otherwise be considered too weak to withstand a conventional treatment regimen.

In some embodiments, the haematopoietic stem and/or progenitor cells are administered as part of an autologous stem cell transplant procedure.

In other embodiments, the haematopoietic stem and/or progenitor cells are administered as part of an allogeneic stem cell transplant procedure.

By “autologous stem cell transplant procedure” it is to be understood that the starting population of cells (i.e. before contact with an agent of the invention) is obtained from the same subject as that to which the final cell population is administered. Autologous transplant procedures are advantageous as they avoid problems associated with immunological incompatibility and are available to subjects irrespective of the availability of a genetically matched donor.

By “allogeneic stem cell transplant procedure” it is to be understood that the starting population of cells (i.e. before contact with an agent of the invention) is obtained from a different subject as that to which the final cell population is administered. Preferably, the donor will be genetically matched to the subject to which the cells are administered to minimise the risk of immunological incompatibility.

Method of Treatment

It is to be appreciated that all references herein to treatment include curative, palliative and prophylactic treatment. The treatment of mammals, particularly humans, is preferred. Both human and veterinary treatments are within the scope of the invention.

Administration

Although the composition for use in the invention can be administered alone, they will generally be administered in admixture with a pharmaceutical carrier, excipient or diluent, particularly for human therapy.

In some embodiments, the composition comprising a combination of a urolithin, a NAD+ precursor, preferably Nicotinamide Riboside and Vitamin B12 is in a combined preparation for simultaneous, separate or sequential use, preferably simultaneous.

In some embodiments, the composition comprising a combination of a urolithin, a NAD+ precursor and Vitamin B12 is in a combined preparation for simultaneous, separate or sequential use with an agent selected from the group consisting of G-CSF analogue, a TPO receptor analogue, and combinations thereof.

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

Dosage

The skilled person can readily determine an appropriate dose of one of the agents of the invention to administer to a subject without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

Subject

In some embodiments, a subject is a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

The invention may be, for example, useful for increasing blood cell production in a subject.

The invention may be, for example, useful for increasing blood cell levels in a subject.

In some embodiments, the subject has or is at risk of having subnormal amounts of haematopoietic cells, for example erythrocytes, leukocytes and/or platelets.

A normal range for leukocytes in humans is 4500-10000 cells/μl. A normal range for erythrocytes in male humans is 5-6 million cells/μl, and in female humans is 4-5 million cells/μl. A normal range for platelets is 140000-450000 per μl. Blood cell levels, which may also be referred to as blood cell counts, may be readily measured by the skilled person using any of a number of techniques known in the art, for example the use of haemocytometers and automated blood analysers.

In some embodiments, a subject has or is at risk of having anaemia, leukopenia and/or thrombocytopenia.

In some embodiments, the subnormal amounts of haematopoietic cells is secondary to a primary or autoimmune disorder of the hematopoietic system, for example congenital bone marrow failure syndromes, idiopathic thrombocytopenia, aplastic anaemia and myelodysplastic syndromes.

Subjects at risk of developing a decrease in blood cell levels include patients suffering from anaemia or myelodysplastic syndromes, those undergoing chemotherapy, bone marrow transplant or radiation therapy, and those suffering from autoimmune cytopenias including but not limited to immune thrombocytopenic purpura, pure red cell aplasia and autoimmune neutropenia.

Subjects at risk of developing post-transplantation complications include haematopoietic cell depleted subjects having received an autologous or allogeneic hematopoietic stem or progenitor cell graft from primary or in vitro manipulated HSPCs.

In some embodiments, the subject may have undergone myeloablative conditioning; chemotherapy; radiotherapy; and/or surgery. The myeloablative conditioning; chemotherapy; radiotherapy; and/or surgery may have resulted in subnormal amounts of haematopoietic cells.

Subjects having or at risk of developing subnormal amounts of haematopoietic cells include subject suffering from blood cancers (e.g. leukaemia, lymphoma and myeloma), blood disorders (e.g. inherited anaemia, inborn errors of metabolism, aplastic anaemia, beta-thalassaemia, Blackfan-Diamond syndrome, globoid cell leukodystrophy, sickle cell anaemia, severe combined immunodeficiency, X-linked lymphoproliferative syndrome, Wiskott-Aldrich syndrome, Hunter's syndrome, Hurler's syndrome, Lesch Nyhan syndrome, osteopetrosis), subjects undergoing chemotherapy rescue of the immune system, and other diseases (e.g. autoimmune diseases, diabetes, rheumatoid arthritis, systemic lupus erythromatosis). Furthermore, subjects having or at risk of developing subnormal amounts of haematopoietic cells include subjects presenting a severe neutropenia and/or severe thrombocytopenia and/or severe anaemia, such as post-transplanted subjects or subjects undergoing ablative chemotherapy for solid tumours, patients suffering toxic, drug-induced or infectious haematopoietic failure (i.e. benzene-derivatives, chloramphenicol, B19 parvovirus, etc.) as well as patients suffering from myelodysplastic syndromes, from severe immunological disorders, or from congenital haematological disorders whether of central (i.e. Fanconi anaemia) or peripheral origin (i.e. G6PDH deficiency).

The invention may be, for example, useful for the treatment or prevention of anaemia, leukopenia and/or thrombocytopenia; an infection (e.g. a non-viral or viral infection); and/or cancer, such as a haematological cancer (e.g. leukaemia, lymphoma or myeloma).

The combination, compositions and cell populations of the invention may be useful in the treatment of the disorders listed in WO 1998/005635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the combination, compositions and cell populations of the invention may be useful in the treatment of the disorders listed in WO 1998/007859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the combination, compositions and cell populations of the invention may be useful in the treatment of the disorders listed in WO 1998/009985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

Methods of Expansion and Culture Media

In another aspect, the invention provides a method of expanding an isolated population of haematopoietic stem and/or progenitor cells (HSPCs) comprising contacting the population with a composition comprising a combination of a urolithin, a NAD+ precursor and Vitamin B12.

In some embodiments, the contacting comprises culturing the population in the presence of the composition comprising a combination of a urolithin, Nicotinamide Riboside and Vitamin B12.

In some embodiments, the method comprises the steps:
(a) providing a population of HSPCs;
(b) optionally culturing the population of HSPCs, preferably in a HSPC expansion or maintenance culture medium;
(c) optionally isolating a sub-population of HSPCs characterised by low mitochondrial membrane potential; and
(d) contacting the population of (a) or (b), or the sub-population of (c) with a composition comprising a combination of a urolithin, a NAD+ precursor and Vitamin B12.

In some embodiments, the population provided in step (a) is obtained from bone marrow, mobilised peripheral blood or umbilical cord blood.

In some embodiments, the product of step (d) is enriched in cells having long-term multi-lineage blood reconstitution capability.

The terms “expansion culture medium” and “maintenance culture medium” as used herein refer to any standard stem cell culture medium suitable for stem cell expansion and maintenance, respectively, such as for example culture media described herein in the examples or described in Boitano et al. (2010) Science 329: 1345-1348.

In another aspect, the invention provides a cell culture medium comprising a composition comprising a combination of a urolithin, a NAD+ precursor, preferably NR and Vitamin B12.

In some embodiments, the culture medium comprises cytokines and growth factors. The cytokines and growth factors can be used with or without supporting stromal feeder or mesenchymal cells, and can comprise, but are not restricted to: SCF, TPO, Flt3-L, FGF-1, IGF1, IGFBP2, IL-3, IL-6, G-CSF, M-CSF, GM-CSF, EPO, oncostatin-M, EGF, PDGF-AB, angiopoietin and angiopoietin-like family including Angl5, prostaglandins and eicosanoids including PGE2, Aryl hydrocarbon (AhR) receptor inhibitors such as StemRegeninI (SRI) and LGC006 (Boitano et al. (2010) Science 329: 1345-1348).

Membrane potential in HSC compartments, in particular mitochondrial membrane potential, can be assayed by methods known to the skilled person, such as described herein in the examples, in particular flow cytometry of cells stained with tetramethylrhodamine methyl ester (TMRM).

Kit

In another aspect, the present invention provides a kit comprising the combination and/or cell populations of the invention.

The cell populations may be provided in suitable containers.

The kit may also include instructions for use.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

Materials and Methods

Flow Cytometry

Flow cytometry analysis was performed on freshly isolated bone marrow (BM) from C57BI6 mice. BM was extracted from crushed femora and tibia. Cell suspension was filtered through a 70 μm cell strainer and erythroid cells were eliminated by incubation with red blood cell lysis buffer (eBioscences). Isolation and stains were performed in ice-cold PBS 1 mM EDTA. Lineage positive cells were then removed with a magnetic lineage depletion kit (BD biosciences). Cell suspensions were then stained with specific antibodies for the stem cell compartment and sorted by FACS (BD FACS Aria III) into 1.5 ml Eppendorf tubes.

Antibodies

The following antibodies were used in this study: rat mAbs against cKit (2B8), Sca1 (D7), CD150 (TC-15-12F12.2), CD48 (HM48-1). The antibodies were purchased from Biolegend, eBiosciences and BD. A mixture of biotinylated mAbs against CD3, CD11b, CD45R/B220, Ly-6G, Ly-6C and TER-119 was used as lineage marker (“lineage cocktail”) and was purchased from BD. DAPI or propidium iodine (PI) staining was used for live/dead cell discrimination.

mHSC Culture

Murine HSCs were sorted into 1.5 ml Eppendorf tubes and were cultured in Stemline II (SIGMA) supplemented with 100 ng/ml SCF (R&D) and 2 ng/ml Flt3 (R&D). This was defined as the basal condition. Different concentrations as indicated of UroA, NR and Vit B12 (MC) were added in specific wells. NR was supplemented every 24 hours.

Analysis of Mitochondrial Activity

Mouse HSCs already in culture were incubated at 37° C. for 1 hour with 200 nM tetramethylrhodamine methyl ester (TMRM; Invitrogen) and 100 nM Mitotracker green. Cells were then washed with FACS buffer and analysed by flow cytometry on a BD LSR II.

Results and Discussion

Combination of UroA NR and Vitamin B12 Induces Lowering of Mitochondrial Membrane Potential

Freshly FACS sorted mHSCs (LKS CD150+CD48−) were cultured in basal media (Stemline+SCF+FLT3L) supplemented with UroA, NR and MC in specific wells (as indicated). Cells were harvested at day 3 and stained with tetramethylrhodamine methyl ester (TMRM; to measure mitochondria membrane potential) and Mitotracker (to measure mitochondrial mass) and analysed by flow cytometry.

We found the proportion of cells in the TMRM^{low} gate we significantly increased and TMRM fluorescent intensity (Mean Fluorescence Intensity, MFI) significantly decreased in the NR and MC condition as compared to the Basal condition. The double combination of NR+MC significantly increased (the proportion of cells in the TMRM^{low} gate) and decreased (MFI TMRM) as compared to NR or MC alone. Intriguingly, the triple combination of UroA NR and MC showed the strongest effect, with significantly higher proportion of cells in the TMRM^{low} gate and significantly lower MFI TMRM compared to the double combination. Mitochondrial mass (measured by Mitotracker) (right panel) decreased significantly with combination of NR+MC and UroA+MC+NR compared to Basal or NR only culture.

In summary, our findings demonstrate the ability of a combination of UroA with Nicotinamide Riboside and Vitamin B12 to synergistically ameliorate HSC function via modulation of mitochondrial membrane potential possibly through mitophagy induction, leading to applications of said combination in the context of HSC transplantation for the treatment of blood malignancies.

All publications mentioned in the above specification are herein incorporated by reference. Various modifications and variations of the disclosed compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. (canceled)

2. The method of claim 17, wherein the urolithin is urolithin A.

3. The method of claim 17, wherein the NAD+ precursor is selected from the group consisting Nicotinic Acid, Nicotinamide, Nicotinamide Riboside (NR), Reduced Nicotinamide riboside (NRH), Nicotinamide Mononucleotide (NMN), Nicotinic acid mononucleotide, Nicotinic acid riboside, and mixtures thereof.

4. The method of claim 17, wherein the combination is Urolithin, Nicotinamide Riboside and Vitamin B12.

5-7. (canceled)

8. A method for use in the treatment or prevention of (a) anaemia, leukopenia and/or thrombocytopenia; (b) an infection; and/or (c) cancer comprising administering a composition comprising a combination of a urolithin, a NAD+ precursor and Vitamin B12 to a subject in need of same.

9. The method according to claim 8, wherein the urolithin is urolithin A.

10. The method according to claim 8, wherein the NAD+ precursor is selected from the group consisting Nicotinic Acid, Nicotinamide, Nicotinamide Riboside (NR), Reduced Nicotinamide riboside (NRH), Nicotinamide Mononucleotide (NMN), Nicotinic acid mononucleotide, Nicotinic acid riboside, and mixtures thereof.

11. The method according to claim 8, wherein the combination is Urolithin, Nicotinamide Riboside and Vitamin B12.

12. The method according to claim 8, wherein the combination is in the form of a pharmaceutical or nutritional composition.

13. The method according to claim 8, wherein a subject has or is at risk of having subnormal amounts of haematopoietic cells.

14. The method according to claim 8, wherein a subject has or is at risk of having anaemia, leukopenia and/or thrombocytopenia.

15. The method according to claim 8, wherein a subject has undergone an intervention selected from the group consisting of a haematopoietic stem cell transplant; a bone marrow transplant; myeloablative conditioning; chemotherapy; radiotherapy; and surgery.

16. The method according to claim 8, wherein the urolithin, the NAD+ precursor and Vitamin B12 is in a combined preparation for simultaneous, separate or sequential use with an agent selected from the group consisting of a G-CSF analogue, a TPO receptor analogue, and combinations thereof.

17. A method of expanding an isolated population of haematopoietic stem and/or progenitor cells (HSPCs) comprising contacting the population with a composition comprising a combination of a urolithin, a NAD+ precursor and Vitamin B12.

18. The method of claim 17, wherein the method comprises the steps:
(a) providing a population of HSPCs;
(b) culturing the population of HSPCs, preferably in a HSPC expansion or maintenance culture medium;
(c) isolating a sub-population of HSPCs characterised by low mitochondrial membrane potential; and
(d) contacting the population of (a) or (b), or the sub-population of (c) with a composition comprising a combination of a urolithin, a NAD+ precursor.

19. A cell culture medium comprising a composition comprising a combination of a urolithin, a NAD+ precursor, preferably Nicotinamide Riboside and Vitamin B12.
